SEQUENCE LISTING (40 sequences)

SEQ ID NO: 1 - protein, 9 aa, Artificial Sequence
  chemically synthesized peptide
VLQMKEEDV

SEQ ID NO: 2 - protein, 9 aa, Artificial Sequence
  chemically synthesized peptide
QMKEEDVLK

SEQ ID NO: 3 - protein, 9 aa, Artificial Sequence
  chemically synthesized peptide
QMEQYIYKR

SEQ ID NO: 4 - protein, 9 aa, Artificial Sequence
  chemically synthesized peptide
GIYIINLKR

SEQ ID NO: 5 - protein, 9 aa, Artificial Sequence
  chemically synthesized peptide
KLLLAARAI

SEQ ID NO: 6 - protein, 10 aa, Artificial Sequence
  chemically synthesized peptide
LLLAARAIVA

SEQ ID NO: 7 - protein, 9 aa, Artificial Sequence
  chemically synthesized peptide
LLAARAIVA

SEQ ID NO: 8 - protein, 9 aa, Artificial Sequence
  chemically synthesized peptide
LAARAIVAI

SEQ ID NO: 9 - protein, 9 aa, Artificial Sequence
  chemically synthesized peptide
AAATGATPI

SEQ ID NO: 10 - protein, 9 aa, Artificial Sequence
  chemically synthesized peptide
TPGTFTNQI

SEQ ID NO: 11 - protein, 9 aa, Artificial Sequence
  chemically synthesized peptide
RLLVVTDPR

SEQ ID NO: 12 - protein, 9 aa, Artificial Sequence
  chemically synthesized peptide
DPRADHQPL

SEQ ID NO: 13 - protein, 9 aa, Artificial Sequence
  chemically synthesized peptide
QPLTEASYV

SEQ ID NO: 14 - protein, 10 aa, Artificial Sequence
  chemically synthesized peptide
PLTEASYVNL

SEQ ID NO: 15 - protein, 9 aa, Artificial Sequence
  chemically synthesized peptide
MLAREVLRM

SEQ ID NO: 16 - protein, 9 aa, Artificial Sequence
  chemically synthesized peptide
LRMRGTISR

SEQ ID NO: 17 - protein, 9 aa, Artificial Sequence
  chemically synthesized peptide
EIEKEEQAA

SEQ ID NO: 18 - protein, 10 aa, Artificial Sequence
  chemically synthesized peptide
EKEEQAAAEK

SEQ ID NO: 19 - protein, 9 aa, Artificial Sequence
  chemically synthesized peptide
KEEQAAAEK

SEQ ID NO: 20 - protein, 9 aa, Artificial Sequence
  chemically synthesized peptide
EEQAAAEKA

SEQ ID NO: 21 - protein, 10 aa, Artificial Sequence
  chemically synthesized peptide
QAAAEKAVTK

SEQ ID NO: 22 - protein, 9 aa, Artificial Sequence
  chemically synthesized peptide
AAAEKAVTK

SEQ ID NO: 23 - protein, 9 aa, Artificial Sequence
  chemically synthesized peptide
VPSVPIQQF

SEQ ID NO: 24 - protein, 18 aa, Artificial Sequence
  chemically synthesized peptide
PRADHQPLTEASYVNLPT

SEQ ID NO: 25 - protein, 22 aa, Artificial Sequence
  chemically synthesized peptide
FREPRLLVVTDPRADHQPLTEA

SEQ ID NO: 26 - protein, 20 aa, Artificial Sequence
  chemically synthesized peptide
GRFTPGTFTNQIQAAFREPT

SEQ ID NO: 27 - protein, 22 aa, Artificial Sequence
  chemically synthesized peptide
EEIEKEEQAAAEKAVTKEEFQG

SEQ ID NO: 28 - protein, 20 aa, Artificial Sequence
  chemically synthesized peptide
TDPRADHQPLTEASYVNLPT

SEQ ID NO: 29 - protein, 21 aa, Artificial Sequence
  chemically synthesized peptide
TWEKLLLAARAIVAIENPADV

SEQ ID NO: 30 - protein, 19 aa, Artificial Sequence
  chemically synthesized peptide
CPRADHQPLTEASYVNLPT

SEQ ID NO: 31 - protein, 23 aa, Artificial Sequence
  chemically synthesized peptide
FREPRLLVVTDPRADHQPLTEAC

SEQ ID NO: 32 - protein, 21 aa, Artificial Sequence
  chemically synthesized peptide
CGRFTPGTFTNQIQAAFREPT

SEQ ID NO: 33 - protein, 23 aa, Artificial Sequence
  chemically synthesized peptide
EEIEKEEQAAAEKAVTKEEFQGC

SEQ ID NO: 34 - protein, 22 aa, Artificial Sequence
  chemically synthesized peptide
TWEKLLLAARAIVAIENPADVC

SEQ ID NO: 35 - protein, 17 aa, Artificial Sequence
  chemically synthesized peptide
CADHQPLTEASYVNLPT

SEQ ID NO: 36 - protein, 20 aa, Artificial Sequence
  chemically synthesized peptide
TDPRADHQPLTEASYVNLPT

SEQ ID NO: 37 - protein, 20 aa, Artificial Sequence
  chemically synthesized peptide
TWEKLLLAARAIVAIENPAD

SEQ ID NO: 38 - protein, 19 aa, Artificial Sequence
  chemically synthesized peptide
GRFTPGTFTNQIQAAFREP

SEQ ID NO: 39 - protein, 17 aa, Artificial Sequence
  chemically synthesized peptide
ADHQPLTEASYVNLPTI

SEQ ID NO: 40 - protein, 21 aa, Artificial Sequence
  chemically synthesized peptide
EEIEKEEQAAAEKAVTKEEFQ

(19) United States
(12) Patent Application Publication
(10) Pub. No.: US 2010/0316574 A1
(43) Pub. Date: Dec. 16, 2010

(54) Oncofetal Antigen/Immature Laminin Receptor Peptides for the Sensitization of Dendritic Cells for Cancer Therapy

(75) Inventor: Eric W. Olle, Unicoi, TN (US)

Correspondence Address: Hogan Lovells US LLP, 1999 AVENUE OF THE STARS, SUITE 1400, LOS ANGELES, CA, 90067, US

(73) Assignee: Quantum Immunologics, Inc., Tampa, FL (US)

(21) Appl. No.: 12/748,250

(22) Filed: Mar. 26, 2010

Related U.S. Application Data
(60) Provisional application No. 61/163,808, filed on Mar. 26, 2009.

(51) Int. Cl. A61K 49/00; C07K 7/06; C07K 7/08; C07K 14/00; C07K 14/765; C07K 16/00; A61K 38/08; A61K 38/16; C07K 2/00; A61K 39/00; A61P 35/00; A61P 37/02; C12N 5/0784; G01N 33/53

(52) U.S. Cl. 424/9.2; 530/328; 530/327; 530/326; 530/350; 530/363; 530/391.1; 514/21.6; 514/21.4; 530/300; 424/185.1; 435/372; 436/501; 435/7.92; 435/7.24; 424/277.1

(57) ABSTRACT

The present invention relates to methods of making peptides or a mixture of peptides that can be used to pulse dendritic cells against the oncofetal antigen/immature laminin receptor protein (OFA/iLRP). More specifically, dendritic cells can be derived from a range of different sources that can direct the immune system to attack specific antigens. Once sensitized, either ex vivo, in vivo or in vitro, the dendritic cells will aid an individual's own immune system to protect against or treat all types of OFA/iLRP-related cancer. The peptides may also be used for detection, diagnosis and monitoring, and treatment of a OFA/iLRP-related cancer.

The present application claims the benefit of the filing date of U.S. Provisional Application No. 61/163,808 filed Mar. 26, 2009, the disclosure of which is incorporated herein by reference in its entirety.

### FIELD OF THE INVENTION

The present invention relates in general to the oncofetal antigen/immature laminin receptor protein (OFA/iLRP). More specifically, the invention provides peptides that can be used for sensitizing dendritic cells for cancer.

### BACKGROUND OF THE INVENTION

The initial characterization of the oncofetal antigen/immature laminin receptor protein (OFA/iLRP) was clone by three independent groups, which studied oncofetal antigen or laminin receptor [1-3]. OFA/iLRP is a highly conserved protein that is over-expressed in a range of different cancers and has a dual function as ribosomal protein p40 [4-27]. The OFA/iLRP protein is comprised of a single polypeptide chain of 295 amino acids and has a molecular weight of about 37-44 KDa. The structure of OFA/iLRP has recently been elucidated to 2.15 Å and shows the region between amino acids 112 to 140 of OFA/iLRP is involved in dimerization [28] of OFA/iLRP for forming laminin receptor protein (LRP). [28]. The mature form of the laminin receptor appears to be a dimer of acetylated immature LRP, with a molecular weight of 67 kDa. Although the mature 67 kDa form is on many normal cells as well as on tumor cells, there appears to be a preferential expression of the OFA/iLRP by fetal and tumor cells. Thus, the expression pattern makes OFA/iLRP a possible candidate protein to sensitize the immune system for the treatment of cancer and other diseases [6]. As such, the use of specific OFA/iLRP peptides in dendritic cell based therapy is a novel application.

Dendritic cells (DCs) are immune cells that form part of the mammalian immune system. Their main function is to process antigenic material and present it on the surface of other cells of the immune system. Thus, they function as antigen-presenting cells. Dendritic cells directly communicate with non-lymph tissue and survey non-lymph tissue for an injury signal (e.g., ischemia, infection, or inflammation) or tumor growth. Once signaled, dendritic cells initiate the immune response by releasing IL-1, TNF.alpha., and various other inflammatory cytokines which trigger lymphocytes and myeloid cells. Various immunodeficiencies, e.g., towards tumors, are thought to result from the loss of dendritic cell function. Dendritic cells have a high capacity for sensitizing MHC-restricted T-cells and provide an effective pathway for presenting antigens to T cells in situ, both self-antigens during T-cell development and foreign antigens during immunity. Thus, there is a growing interest in using dendritic cells ex vivo as tumor or infectious disease vaccine adjuvants. Dendritic cells can be derived from a range of different sources (myeloid and lymphoid) that can direct the immune system to attack specific antigens. For example, they may be derived from the following sources: monocyte derived (CD14+), hematopoietic stem cell derived (CD34+ or CD133+ or CD117+), Plasmacytoid (CD303+/CD304+), Myeloid derived (CD1c+ or CD141+ or CD209+), or Langerhans cells. Once sensitized, either ex vivo, in vivo or in vitro, the dendritic cells will aid an individual's own immune system to protect against or treat all types of OFA/iLRP-related diseases or cancers. Sensitization, or pulsing of dendritic cells is a process by which dendritic cells are exposed to a target protein in order to elucidate a targeted immune response.

OFA/iLRP dendritic cell therapy has been shown to increase the survival of patients with late-stage carcinomas with minimal side effects [13, 22]. Previous experiments used peptides with a putative MHC (major histocompatibility complex) binding sequence to sensitize the dendritic cells. However, the putative MHC binding site can be missed by the immune system because of its location on the peptide or because it spans more than one peptide. Siegel, et al., used peptides which targeted HLA-A*201, but did not take into account the peptide solubility and other problems associated with peptides [22]. Rohrer, et al., analyzed the proliferation profile of amino acids that overlapped an OFA/iLRP 12-mer peptide derived from mouse [29]. This study focused on the analysis of the ratio X/n where X is the length of the protein in amino acids and the n is the peptide length (FIG. 1). However, based on this sequential peptide method, the putative HLA (human leukocyte antigen) site can still be missed or not recognized by the immune system due to various reasons, including, but not limited to: peptide solubility, peptide structure, HLA sites located between two peptides, HLA sites flanked by amino acids that restrict HLA binding and improper secondary structure. Therefore, the use of sequential peptides, while informative, has a high probability of missing useful protein sequences that could be recognized if the full-length protein was processed. In fact, previous work has failed to show several of the putative HLA binding sequences that were found by combining publicly available HLA binding sequence prediction programs and using statistical methods to find areas that are more likely to generate an immune response.

Additionally, previous OFA/iLRP cell-based therapy used bacterially expressed or small HLA specific peptides. [13, 22]. Bacterial expression is time-consuming and difficult to produce in a GMP-certified manner. Therefore, a need exists for developing peptides specifically designed with regions with an increased number of putative MHC binding sites for dendritic cell and immune system stimulation. A need also exists for developing effective OFA/iLRP dendritic cell therapy using peptides.

### SUMMARY OF THE INVENTION

One aspect of the present invention provides isolated peptides or mixtures thereof that can sensitize dendritic cells. Examples of the peptides include, but are not limited to: VLQMKEEDV, QMKEEDVLK, QMEQYIYKR, GIYIINLKR, KLLLAARAI, LLLAARAIVA, LLAARAIVA, LAARAIVAI, AAATGATPI, TPGTFTNQI, RLLVVTDPR, DPRADHQPL, QPLTEASYV, PLTEASYVNL, MLAREVLRM, LRMRGTISR, EIEKEEQAA, EKEEQAAAEK, KEEQAAAEK, EEQAAAEKA, QAAAEKAVTK, AAAEKAVTK, VPSVPIQQF, and mixtures thereof.

Additional amino acids may be added to either the n-terminal and/or the c-terminal of the peptides or the mixture of the peptides. In one embodiment, the peptides of the present invention include the following peptides:

PRADHQPLTEASYVNLPT (129),
FREPRLLVVTDPRADHQPLTEA (117),
GRFTPGTFTNQIQAAFREPT (101),
EEIEKEEQAAAEKAVTKEEFQG (208),
TDPRADHQPLTEASYVNLPT (129-a)
or
TWEKLLLAARAIVAIENPADV (54).

In another embodiment, the peptides of the present invention are derived from the dimerization region of OFA/iLRP. The peptides can sensitize dendritic cells.

The peptides of the present invention may be conjugated to a carrier that increases the peptide's immune stimulation, stability, and/or solubility. Examples of the carriers include, but are not limited to, keyhole limpet hemocyanin (KLH), serum albumin, biological polymers, antibody, chemotherapy, carbon nano-tubes, microelectro/electrofluidic device, molecular machine, amino acid MAP polymer, biologically active lipids, biologically active sugar molecules/polymers, and colloidal particles.

The peptides of the present invention may also be modified to include acetylation, fatty acidification, myristic acidification, palmitoylation, benzyloxycarbonylation, abidation, p-Nitroanilide, AMC, succinylation, NHS, CMK/FMK, D-amino acids, dinitrobenzoylation, methylation, phosphorylation, AHX, SO3H2, octanoic acid, biotin, FITC, GAM, Dansyl, MCA, HYNIC, DTPA, cyclic formations, or a multiple antigenic peptide system (MAP).

Another aspect of the present invention provides a composition that includes the peptides of the present invention. The composition can be a pharmaceutical composition or a vaccine. The pharmaceutical composition may include a pharmaceutically acceptable carrier. The composition may also be a dendritic cell that is sensitized by the peptides of the present invention.

The present invention also provides a method of treating a subject with OFA/iLRP-related cancer. The method includes the step of administering the peptides of the present invention, either individually or as a mixture, to the subject in an amount that is sufficient to decrease the progression of the OFA/iLRP-related cancer. In one embodiment, the peptides induce an immune response in the subject that decreases the progression of the OFA/iLRP-related cancer.

In another embodiment, the method of treating a subject with OFA/iLRP-related cancer includes the steps of (a) sensitizing dendritic cells with peptides of the present invention, (b) administering the sensitized dendritic cells to the subject in an amount that is sufficient to induce an immune response that decreases the progression of the OFA/iLRP-related cancer.

Peptides of the present invention may also be used in a method for determining the amount of an antibody against OFA/iLRP in a sample. The method comprises:
(a) contacting peptides of the present invention with the sample under a condition that allows the antibody to bind to the peptide to form a complex,
(b) determining the amount of complex formed in the sample.

In another embodiment, the peptides of the present invention are used in a method for monitoring the progress of a OFA/iLRP related cancer vaccination therapy in a subject. The method comprises the steps of (1) administering the peptide of the invention to a site of the subject subcutaneously or intradermally in an amount that is sufficient to detect the immune response of the subject to the therapy, (2) monitoring the diameter of the reaction at the site of administration.

In a further embodiment, the peptides of the present invention are used in a method for ex vivo monitoring the progress of an OFA/iLRP-related cancer treatment in a subject. The treatment may induce either a T-cell related response or a B-cell related response (the antibody response). The method comprises the steps of (1) providing a biofluid of the subject that receives the treatment, (2) contacting the peptides of the present invention with the biofluid under a condition that allows the interaction of the peptides with the T-cell or the B-cell or the products generated by the T-cell or B-cell, (3) determining the amount of interaction by ELISA, fluorescent polarization, resonance, or FACS method.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope

### BRIEF DESCRIPTION OF THE FIGURES

FIG. 1. Computer analysis of OFA/iLRP and HLA binding motifs. (A) Graph of the number of amino acids as a function of predictions. (B) Graph of the number of amino acids as a function of % OPT. (C) Graph of the number of distribution sites as a function of the 5 amino acid bin.

FIG. 2. Whisker plot of the selected peptides for dendritic cell sensitization. Graph of clustered HLA binding peptides as a function of % OPT. The solid circle shows outliers that can skew results and allows for a mis-representation of where the strongest immunogenic regions are found.

FIG. 3. Fluorescent Activated Cell. Sorting (FACS) analysis of pulsed dendritic cells. (A) FACS of dendritic cells pulsed with full-length recombinant human OFA. (B) FACS of dendritic cells pulsed with a peptide mixture. (C) Graph of the effect of pulsing agents on dendritic cell recognition of the 129 peptide.

FIG. 4. Effect of peptide modification on dendritic cell recognition of the 129 region. (A) FACS of the 129 peptide. (B) FACS of the 129a peptide. (C) Median fluorescence intensity (MFI) of the 129 and 129a regions. (D) Graph of the effect of pulsing agents on dendritic cell recognition of the 129 peptide versus the 129a peptide.

FIG. 5. Affect of OFA/iLRP peptides on cell adhesion. (A) Graph of DU-145 cell adhesion. (B) Graph of SK-MEL cell adhesion.

FIG. 6. Affect of OFA/iLRP peptides on cell viability. (A) Graph of cell viability for peptide 1, with and without laminin. (B) Graph of cell viability for peptide 2, with and without laminin. (C) Graph of cell viability for peptide 3, with and without laminin.

### DETAILED DESCRIPTION OF THE INVENTION

One aspect of the present invention provides peptides or a mixture of peptides that can be used to pulse dendritic cells against the OFA/iLRP. The peptides, which direct the immune system to recognize specific regions of the OFA/iLRP protein, are designed around a range of unique protein regions such as: homodimer formation region, laminin-binding regions, multi-drug resistant regions, ribosomal interaction regions, and other sites of biological significance. The peptides are designed based on the full-length protein, with a focus on peptides that are specific to OFA/iLRP dimer formation, antigenicity, MHC-1 binding, MHC-2 binding, proteasome cleavage, solvent accessibility, and protein sequence. The present invention uses computer and statistical analysis to determine optimal peptides that may be used against OFA/iLRP-related disease therapy. This method allows for the computational analysis of X-n peptides and the addition of rapid analysis of multiple peptide lengths, which increases the probability of developing optimal peptides for dendritic cell or vaccination therapy using OFA/iLRP or any other possible protein used in similar treatment/therapies.

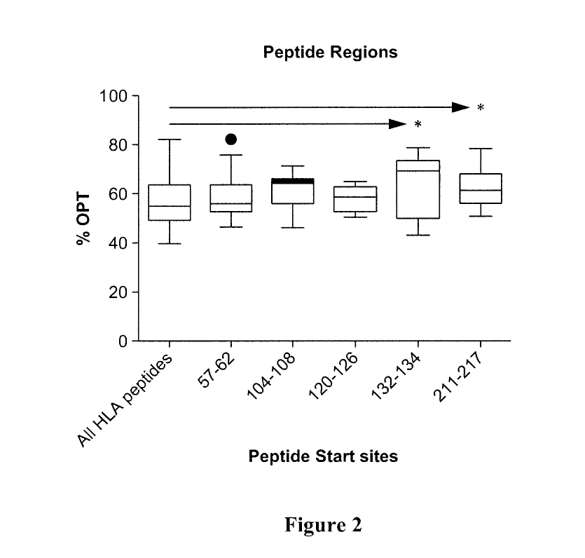

To determine the distribution of different OFA/iLRP epitopes, the OFA/iLRP protein sequence was mined for all known HLA binding motifs using SYFPEITHI (Hans-Georg Rammensee, Jutta Bachmann, Niels Nikolaus Emmerich, Oskar Alexander Bachor, Stefan Stevanovic: SYFPEITHI: database for MHC ligands and peptide motifs. Immunogenetics (1999) 50: 213-219 (access via : www.syfpeithi.de)). However, instead of analyzing HLA binding motifs individually, a table of all known HLA binding motifs in the database were stored in Excel with the sequence, starting site, and the % optimal binding (% match to experimental HLA binding data). Once the data for the HLA binding sites were obtained, they were analyzed using Prism 5.0 for Mac OS X (GraphPad Software, Inc.). To look for regions with increased HLA binding sites, several different analyses were done. First, the starting site of the predicted HLA binding site was plotted to show potential regions of increased HLA binding probability. Then, the % optimal (OPT) was examined and plotted against the amino acid starting site. Finally, to look for regions of increased numbers of HLA binding sites, the number of sites were binned (5 amino acids). These analyses together indicated that there were specific regions that had a greater distribution of HLA binding sites. Once the regions with a high number of HLA binding sites were identified, the data from the Excel sheet in these regions was extracted and the % OPT was plotted against all of the predicted HLA binding regions. To analyze regions with significant differences to the entire protein (all HLA sites), the selected peptide regions were analyzed using a one-way ANOVA, followed by a Dunnets multiple comparison tests. The analysis showed that there was a significant difference between the groups (p=0.0068). The Dunnets post-hoc analysis showed that the 132-134 and the 211-217 peptide groups were significantly different than the control group of all MHC peptides (*=p<0.05) (FIG. 2).

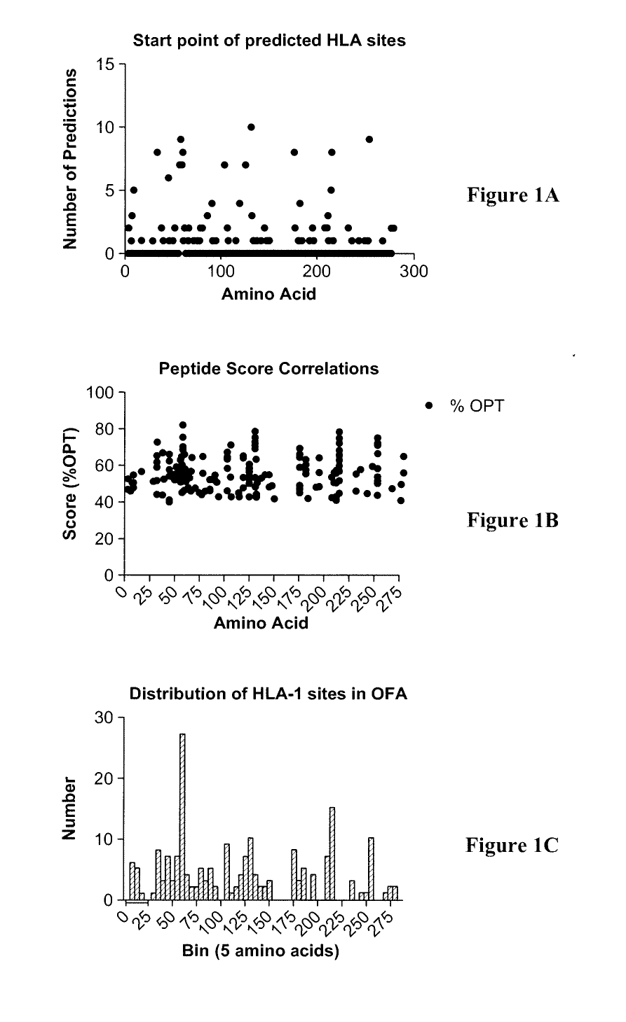

Using the mining methodology of the present invention described herein, peptides that can be used to sensitize the immune system were identified. Table 1 provides a list of examples of the peptide sequences of the present invention. Analyzing the number of times a region can putatively bind MHC proteins, peptide regions can be selected and/or combined to develop bioactive peptides (FIG. 1). Using the distribution analysis along with the mean % OPT score for a region, five regions were found to have a higher probability of binding the appropriate components of the immune system. This allows for sensitization of patients using a range of different ex vivo, in vitro or in vivo stimulation to treat OFA/iLRP-related diseases.

Accordingly, in one embodiment, the peptides of the present invention include, but not limited to: VLQMKEEDV, QMKEEDVLK, QMEQYIYKR, GIYIINLKR, KLLLAARAI, LLLAARAIVA, LLAARAIVA, LAARAIVAI, AAATGATPI, TPGTFTNQI, RLLVVTDPR, DPRADHQPL, QPLTEASYV, PLTEASYVNL, MLAREVLRM, LRMRGTISR, EIEKEEQAA, EKEEQAAAEK, KEEQAAAEK, EEQAAAEKA, QAAAEKAVTK, AAAEKAVTK, VPSVPIQQF, and mixtures thereof. Additional amino acids may be added to either the n-terminal and/or the c-terminal of the peptides or the mixture of the peptides.

In another embodiment, the peptides of the present invention are derived from the dimerization region of OFA/iLRP.

The peptides of the present invention may be conjugated with appropriate carriers for immune stimulation, stability, and/or peptide solubility. The types of conjugation include, but are not limited to: keyhole limpet hemocyanin (KLH), serum albumin, biological polymers, antibody, chemotherapy, carbon nano-tubes, microelectro/electrofluidic device, molecular machine, amino acid MAP polymer, dendromers, biologically active lipids, biologically active sugar molecules/polymers, colloidal particles, other peptide sequences and completed with a range of proteins. The location of the cysteine residue for conjugation can occur at either terminus or internally as needed. The goal of conjugation is to increase the immune system, anti-cancer as a treatment, or any other relevant activity related to the OFA/iLRP peptides.

The peptide sequences of the present invention may vary slightly to allow for greater immune system reactivity, increased solubility, and other functions, and such variations of the peptide sequences are considered part of the peptides of the present invention. Additionally, one or more immune reactive peptide sequences of the present invention may be placed together to form a new immune reactive peptide. The sequences can be from regions of highly predictable immune activity or from several other sites or proteins. Additionally, the peptides may be used to block or enhance specific functions that are not related to the immune system. A concatenated peptide of one or more repeated peptides may be made to form a long biologically active polymer. During the manufacture, the peptides may have one or more modifications, including but not limited to: acetylation, formulation, fatty acidification, myristic acidification, palmitoylation, benzyloxycarbonylation, abidation, p-Nitroanilide, AMC, succinylation, NHS, CMK/FMK, D-amino acids, dinitrobenzoylation, methylation, phosphorylation, SO3H2, octanoic acid, biotin, FITC, GAM, Dansyl, MCA, HYNIC, DTPA, cyclic formations, multiple antigenic peptide system (MAP), and/or others that affect OFA/iLRP peptide function, including to increase solubility, stability, immune reactivity, and/or biological activity.

The peptide(s) of the present invention can be directed to specific regions of the OFA/iLRP that may decrease non-specific effects during the treatment of OFA/iLRP-related diseases or vaccine. The peptides are made from one peptide sequence or a combination of peptide sequences shown in Table 1. To determine which regions have an increased probability of immune system stimulation, clustered putative HLA binding sites were designed and the mean % OPT (the score of the peptide when compared to the consensus) [53, 54] was compared to all putative HLA sites across OFA/iLRP (FIG. 2). An example of the peptides specifically designed to include more than one putative HLA binding site that were used for the comparison are listed in FIG. 2.

An individual or a combination of peptides of the present invention may be used to sensitize the immune system with the provided OFA/iLRP sequences. The peptides designed to induce an immune response against OFA/iLRP may also be used to provide additional clinical applications, including but not limited to: receptor binding, blocking Laminin function and/or affecting other OFA/iLRP cellular functions. The final product may be conjugated to increase immune reactivity of the OFA/iLRP peptides. The conjugations may be formed via different methods described herein or known in the art, including the addition of cysteine to react to a maledioamide KLH protein [55, 56]. These peptides and combinations thereof, may be conjugated or modified prior to use as an ex vivo, in vivo, or in vitro vaccination against cancer.

According to embodiments of the present invention, the computer-based approach identified several possible protein sequences that may be used for dendritic cell and immune system therapy. In one embodiment, the mean % OPT was calculated for three peptides designed around amino acids 132, 117 and 54 (FIG. 2). The designed peptides were compared to the mean % OPT score, and all of them are above the mean with the 132 region being significantly different (FIG. 2). The peptides have a flanking sequence of at least three amino acids that are not part of the HLA prototypical sequence and can be modified to optimize antigen processing and HLA binding if needed. Other possible peptides start around amino acids 104 and 211 and can be used alone or in combination with other peptides.

The peptide(s) of the present invention can be used to replace the bacterially expressed OFA/iLRP during ex vivo dendritic cell induction and sensitization. The peptide(s) can be conjugated to various macromolecules, provided they are appropriate for human or veterinary applications.

The peptides may be prepared by chemical synthesis or biochemical synthesis using Escherichia coli or the like. Methods well known to those skilled in the art may be used for the synthesis.

When the peptide of the invention is chemically synthesized, methods well known in the field of peptide synthesis may be used. For example, such methods as the azide method, the acid chloride method, the acid anhydride method, the mixed acid anhydride method, the DCC method, the active ester method, the carbodiimidazole method and the oxidation-reduction method may be enumerated. Either solid phase synthesis or liquid phase synthesis may be used. A commercial peptide synthesizer (e.g., Shimadzu PSSM-8) may also be used.

After the reaction, the peptide(s) of the invention may be purified by a combination of conventional purification methods such as solvent extraction, distillation, column chromatography, liquid chromatography or re-crystallization.

According to one aspect of the present invention, the peptides may be included in a composition to be administered to a subject. The composition can be a pharmaceutical composition or a vaccine. The pharmaceutical composition may include a pharmaceutically acceptable carrier. The composition may also be a dendritic cell that is sensitized by the peptides of the present invention.

As used herein, dendritic cells (DCs) are immune cells that process antigen material and present it on the surface to other cells of the immune system, thus functioning as antigen-presenting cells. Different processes may be used to sensitize the dendritic cells to antigens. In one embodiment, these processes comprise a step of placing the dendritic cells in contact with antigenic peptides (“peptide pulsing”). This approach consists of incubating the dendritic cells for a variable time (usually from about 30 minutes to about 5 hours) with one or more antigenic peptides, i.e., with a peptide derived from an antigen so that the treatment with the peptides will result in an antigen-presenting cell, which is also called a sensitized dendritic cell.

Treatment of the dendritic cells with the cancer-specific antigen can be by any method which results in the dendritic cells presenting the antigen so as to stimulate host immunity when a vaccine composition or a composition comprising the peptides is administered to the mammal, e.g., by pulsing or culturing the dendritic cells in the presence of the antigen prior to administration of the vaccine composition to the mammal.

Dendritic cells can be administered to the mammal by any method which allows the dendritic cells to reach the appropriate cells. These methods include, e.g., injection, infusion, deposition, implantation, oral ingestion, or topical administration, or any combination thereof. Injections can be, e.g., intravenous, intramuscular, intradermal, subcutaneous or intraperitoneal. Single or multiple doses can be administered over a given time period, depending upon the cancer, as can be determined by one skilled in the art without undue experimentation. The injections can be given at multiple locations. Administration of the dendritic cells can be alone or in combination with other therapeutic agents.

As used herein, “vaccine” means an organism or material that contains an antigen in an innocuous form. The vaccine is designed to trigger an immunoprotective response. The vaccine may be recombinant or non-recombinant. When inoculated into a non-immune host, the vaccine will provoke active immunity to the organism or material, but will not cause disease. Vaccines may take the form, for example, of a toxoid, which is defined as a toxin that has been detoxified but that still retains its major immunogenic determinants; or a killed organism, such as typhoid, cholera and poliomyelitis; or attenuated organisms, that are the live, but non-virulent, forms of pathogens, or it may be antigen encoded by such organism, or it may be a live tumor cell or an antigen present on a tumor cell.

While the dosage of the vaccine composition depends upon the antigen, species, body weight of the host vaccinated or to be vaccinated, etc., the dosage of a pharmacologically effective amount of the vaccine composition will usually range from about 50 .mu.g to about 500 .mu.g per dose, per kilogram of body weight, in a mouse model.

As a general rule, the vaccine composition of the present invention is conveniently administered orally, parenterally (subcutaneously, intramuscularly, intravenously, intradermally or intraperitoneally), intrabuccally, intranasally, or transdermally. The route of administration contemplated by the present invention will depend upon the antigenic substance and the co-formulants.

The dosage of the vaccine composition will be dependent upon the selected antigen, the route of administration, species, body weight, and other standard factors. It is contemplated that a person of ordinary skill in the art can easily and readily titrate the appropriate dosage for an immunogenic response for each antigen to achieve the effective immunizing amount and method of administration.

The composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Compositions suitable for injectable use include sterile aqueous solutions (where water soluble) or dispersions and sterile powders for the extemporaneous preparation of sterile injectable solutions or dispersion. For intravenous administration, suitable carriers include physiological saline, sterile water, Cremophor EL™ (BASF, Parsippany, N.J.), or phosphate buffered saline (PBS). In all cases, the composition must be sterile and should be fluid to the extent that easy syringeability exists. It should be stable under the conditions of manufacture and storage, and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars or polyalcohols such as manitol, sorbitol, or sodium chloride, in the composition. Prolonged absorption of the injectable compositions can be brought about by including in the composition an agent which delays absorption, for example, aluminum monostearate and gelatin.

Sterile injectable solutions can be prepared by incorporating the compounds in the required amounts in an appropriate solvent with one or a combination of ingredients enumerated above, as required, followed by filtered sterilization. Generally, dispersions are prepared by incorporating the compounds into a sterile vehicle which contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally include an inert diluent or an edible carrier. For the purpose of oral therapeutic administration, the compounds can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compositions are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

The compositions of the invention can also be prepared in the form of suppositories (e.g., with conventional suppository bases such as cocoa butter and other glycerides) or retention enemas for rectal delivery.

In one embodiment, the compositions are prepared with carriers that will protect the compounds against rapid elimination from the body, such as a controlled-release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of active compound calculated to produce the desired therapeutic effect in association with the required pharmaceutical carrier.

The compositions of the invention, can be included in a container, pack, or dispenser together with instructions for administration to form packaged products. Other active compounds can also be incorporated into the compositions.

The invention also provides pharmaceutical compositions comprising the peptides of the present invention and pharmaceutically acceptable carriers or excipients. Pharmaceutically acceptable excipients are known in the art, and are relatively inert substances that facilitate administration of a pharmacologically effective substance. For example, an excipient can give form or consistency, or act as a diluent. Suitable excipients include, but are not limited to, stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

The present invention provides a method of using the peptides of the present invention for detection, diagnosis and monitoring, and treatment of a OFA/iLRP-related cancer. For the purpose of the present invention, a OFA/iLRP-related cancer refers to any disease, disorder, or condition associated with the epitope expression of OFA/iLRP (either increased or decreased relative to a normal sample, and/or inappropriate expression, such as presence of expression in tissues(s) and/or cell(s) that normally lack the epitope expression).

In one embodiment, the present invention provides a method of treating a subject with OFA/iLRP-related cancer. The method includes the step of administering the peptides of the present invention, either individually or as a mixture to the subject in an amount that is sufficient to decrease the progression of the OFA/iLRP-related cancer. The peptides may induce an immune response in the subject that decreases the progression of the OFA/iLRP-related diseases. The immune response may include both T-cell or B-cell related response. The peptide may also induce a response that is independent of immune response. An example of this response could be but not limited to: viability, adhesion, migration, vascularization, or other responses that maybe independent of the immune response.

In another embodiment, the method of treating a subject with OFA/iLRP-related cancer includes the steps of (a) sensitizing dendritic cells with peptides of the present invention, (b) administering the sensitized dendritic cells to the subject in an amount that is sufficient to induce an immune response that decreases the progression of the OFA/iLRP positive cancer. Dendritic cell sensitization and administration are discussed above and will not be repeated herein. Examples of dendritic cells include, but are not limited to: monocyte derived cells (CD14+), hematopoietic stem cell derived cells (CD34+ or CD133+ or CD117+), plasmacytoid (CD303+/CD304+), myeloid derived cells (CD1c+ or CD141+ or CD209+), or Langerhans cells.

Peptides of the present invention may also be used in a method for determining the amount of an antibody against OFA/iLRP in a sample. The method comprises:
(a) contacting peptides of the present invention with the sample under a condition that allows the antibody to bind to the peptide to form a complex,
(b) determining the amount of complex formed in the sample.
   Conventional methods may be used to determine the amount of the complex with an antibody. Examples include, but not are limited to, ELISA, fluorescent polarization, resonance, FACS, or any known methods capable of detecting antibodies.

In another embodiment, the peptides of the present invention are used in a method for monitoring the progress of a OFA/iLRP-related cancer vaccination therapy in a subject. The method comprises the steps of (1) administering the peptide of the invention to a site of the subject subcutaneously or intradermally in an amount that is sufficient to detect the immune response of the subject to the therapy, (2) monitoring the diameter of the reaction at the site of administration.

In further embodiment, the peptides of the present invention are used in a method for ex vivo monitoring the progress of a OFA/iLRP-related cancer treatment in a subject. The treatment is capable of inducing either a T-cell related response or a B-cell related response such as an antibody response. The method comprises the steps of (1) providing a biofluid of the subject that receives the treatment, (2) contacting the peptides of the present invention with the biofluid under a condition that allows the interaction of the peptides with the T-cell or the B-cell or the products generated by the T-cell or B-cell, (3) determining the amount of interaction by ELISA, fluorescent polarization, resonance, or FACS method.

For the purpose of the present invention, a biofluid is any biological fluid or tissue lysate that can be excreted, secreted, obtained with a needle, or developed as a result of a pathological process. Examples of a biofluid include, but are not limited to, blood, urine, tissue lysate, serum, plasma, bile, sweat, saliva, cyst fluid, blister fluid, abcess fluid, cerebrospinal fluid, or other.

Products of the T-cell include, but are not limited to, IL-2, IFN-gamma, TNF-alpha, IL-4, IL-6, IL-17A, IL-10, T-cell receptors, chemokines, perorin, granzyme b, IL-9, IL-1beta, GM-CSF, TGF-beta, CD4, CD8, integrins, MHC or others.

Products of the B-cell include, but are not limited to immunoglobulin, BLAME, BTC, HVEM/TNFRSF14, IFNGR2, IgG, IgM, IL-10, IL-13 integrins, DLF, LAX, leukotriene, Lyn, Lillrc1, NFAM1, NTB-a, OX40L, Pax5, PDCD6, WSX-1/IL-27R, TER-119, TRA, TREML2, TSLP, Vav-1, B-cell receptors, BAFF, CD79A, CD40 ligand, BCL-6, ADAM, IL-11, IL-4, CD27, STAT and others.

Peptides of the present invention have many applications. Some of the applications are listed below as examples.

### Use of Peptides In Dendritic Cell Therapy

Peptides of the present invention can be used as part of dendritic cell therapy or ex vivo immune therapy. OFA/iLRP peptide(s), alone or in conjunction with KLH, or other immune system stimulants, adjutants or other molecules can be used to elicit an ex vivo immune response. “Pulsed” dendritic cells are injected back into the donor to provide anticancer immune response that may be able to decrease the progression of all forms of OFA/iLRP positive cancer. The initial effect of the peptides conjugated to KLH can be measured in cell culture using cytokine/chemokine expression.

The OFA/iLRP peptides to be used for sensitization are prepared with the generation of dendritic cells from monocytes starting with either monocytes isolated from peripheral blood, leukapheresis cells, or buffy coats [30, 31]. The isolation and culture of the monocytes follow standard protocols using complete RPMI-10 (RPMI 1640 with fetal bovine serum, 15 mM HEPES and 1× antibiotic/antimycotic solution or similar) [30-35]. The differentiation of the monocytes and pulsing (exposure to the antigen) will be done according to standard methods [13]. As a measure of dendritic cell sensitization and maturation, the production of gamma-interferon (γ-IFN) can be measured in the cell culture medium. An increase in y-IFN in the cell culture medium that is statistically greater than un-stimulated control cells is considered positive dendritic cell stimulation by an OFA/iLRP peptide. Previous studies have used a combination of several different cytokines, and multiple cytokines or chemokines, may be assayed to provide a complete cytokine landscape. Additional analysis using cluster of differentiation (CD) molecule expression to determine the subset of immune cells before, during, and after cell stimulation may be performed. An animal model may be used to determine the exact effect of the different peptides on the overall in vivo cytokine landscape post-injection. This will also provide proof of concept for scaling to human treatment. The combination of several model systems (i.e., cell culture, healthy donor whole blood, and animal models) will provide data to develop the OFA/iLRP peptide models to human anti-cancer therapy.

Peptides have been used before to pulse human dendritic cells [23]; however, the regions used are different with one exception. The previous work used a peptide that started at amino acid 58 [23], whereas the one here starts at amino acid 54 for very specific reasons. First, the start site was chosen for better solubility and other problems associated with three non-polar, hydrophobic residues [23]. The next amino acid has a non-polar side chain, therefore, the threonine was, chosen for the initial amino acid. Additionally, the lack of charge and the change in the sequence may be enough to affect binding which in turn can affect the potential for MHC binding [53, 54, 57].

### Use of Peptides as a Dendritic Cell Patient Immune Response Tool

According to another aspect of the present invention, the peptides of the present invention, for examples, the peptides listed in Table 1, can be used to monitor the progress of a cancer vaccination therapy using OFA/iLRP. In one embodiment, the individual fragments can be injected subcutaneously or intradermally to monitor the immune response of a patient. After injection, the site is monitored for response and the diameter of reaction is measured. If more than one peptide is used for sensitization, there can be multiple injection sites and the reactions can be compared. If the peptides were conjugated with KLH or similar substance, the conjugate alone can be used as a positive control for immune response. When there are more than two or three sensitizing peptides/reagents to be tested, the peptides can be used on a modified skin scratch test, skin prick test, or skin patch test similar to the Mantoux/PPD test [36].

The peptides to be used for the skin prick test may be prepared in a range of dilutions and with a range of different peptides from OFA/iLRP, KLH (or other conjugate), or any other protein used for dendritic cell/cancer vaccination therapy [13, 27]. The dilutions and/or different peptides can be used to map immune responses of an individual and used to sensitize the immune system. If a protein, in lieu of a peptide, is used to sensitize a patient, the modified skin patch/prick test can be used to find the immune reactive epitopes of the individual. Peptides (6 to 30 amino acids) synthesized to cover the putative MHC-1 or 2 of the protein may be used to sensitize/vaccinate the patient. The peptides can be chosen in a manner similar to the selection of OFA/iLRP peptides. Alternatively, a library of peptides that cover the protein sequence can be synthesized and used to determine the epitope and amount of reaction. The immune response to peptides and control solutions can be concurrently determined by scratching/pricking the skin with available skin patch test kit applicators. Alternatively, a needle can be “loaded” with the appropriate peptide or control solution and a patch of skin can be tested. The immune reaction may appear in as quickly as 20 minutes. However, the reaction will probably occur over a period of 1 week after inoculation, although the maximal hypersensitivity reaction will tend to occur between 24-72 hours post-inoculation. Once the reaction and optimal dilution of the peptides are determined, clinicians may determine the size or grade of dendritic cell or vaccination therapy using a standardized test. This test is similar to the tuberculin or Mantoux/PPD test for tuberculosis [36]. The information provided by the delayed hypersensitivity reaction will indicate that the dendritic cell or vaccination was effective and how sensitive the patient is to the target epitopes.

In another embodiment, the peptides of the present invention may be used for determining the extent of immune reactivity caused by the dendritic cell or vaccination therapy by ex vivo quantification of cytokine response. To perform the measurement of the ex vivo cytokine response, the patient's blood is drawn and white blood cells are isolated via metrizamide gradient, Ficoll gradient, or hypotonic lysis of red blood cells. The resultant WBC is washed and plated in an appropriate growth medium. The cells are incubated at 37° C. in a humid environment containing 5%. CO2. After 18-24 hours, the cells are grown in either individual plates or multi-well plates that can be “challenged” using a range of dilutions of peptides from OFA/iLRP or any other dendritic cell or vaccination therapy. After incubating as above for up to 72 hours, the cell culture medium is isolated and the cytokine/chemokine expression is determined using standard ELISA or multiplex ELISA technologies. Some of the cytokine expression that can be used to determine immune activity include, but are not limited to: GM-CSF, IFN-γ, IL-4, IL-10, TGF-α, TNF- [symbol] , IL-6, IL-2, and/or IL-12. Several other commonly used techniques may be applied to determine the immunological responses of OFA/iLRP and OFA/iLRP peptide therapy [37-40].

### Use of Peptides as a Vaccine

The peptides of the present invention, for example, the peptides listed in Table 1, can be used for an in vivo vaccine, individually or in a conjugated state. They can be directly injected into individuals in order to provide a protection against cancer (including pre-diagnosis) or to help slow the progression of present cancer. The peptides are conjugated to appropriate substrates to confer a proper in vivo vaccination response. Individuals are exposed to the OFA/iLRP peptides, either intramuscularly, intradermally, intravascular, orally, or via any other commonly used route/mechanism. Once injected, the individual's immune system mounts an appropriate immune response to protect against OFA/iLRP-positive cancer and possibly other OFA/iLRP-related diseases. The vaccination is useful to: (i) decrease the probability of having OFA/iLRP-positive cancers; (ii) decrease the rate of recurrence once treated for a localized form of OFA/iLRP-positive cancers; (iii) aid in the augmentation of current chemotherapeutic, radionucleotide-seeding, or radiation-based cancer therapies; (iv) aid in slowing the progression of advanced stage cancer; (v) augment immunity via OFA/iLRP-sensitized dendritic cell therapy.

The peptides can be injected into animals, to sensitize against OFA/iLRP after a series of sensitizations, and/or a minimal titer against OFA/iLRP. As a control for non-specific immune system affects, control animals may be sensitized against keyhole limpet hemocyanin. After the appropriate series of sensitizations, the animals can be “challenged” by injecting OFA/iLRP positive cancer cells into the tail vein of the sensitized animals. The injected cancer cells will colonize in the lungs of the animals. The animals sensitized against OFA/iLRP should have a lower number of cancer colonies in the lungs than the untreated animals. Alternatively, other animal models/metrics can be used. However, a system where immuno-compromised animals are used may not work due to the nature of the treatment. The problem with the majority of cancer models in animals is that they use SCID, hematological in origin, or other systems that lack a fully functional immune system. However, OFA/iLRP cancer therapy has been successfully modeled in animals before [6, 9, 11, 18, 23, 41-43]. Due to the immune nature of this treatment, a functional immune system is needed. Alternative methods exist to determine the therapeutic value of these peptides against OFA/iLRP-positive cancers. Several other commonly used techniques may be applied to determine the immunological responses of OFA/iLRP and OFA/iLRP peptide therapy [37-40], the relevant content of which are incorporated herein by references.

### Use of Peptides For the Treatment of OFA/iLRP Associated Diseases

The peptides of the present invention, for example, those listed in Table 1, individually or in combination, can be used in a conjugated or unconjugated form to alter the progression of diseases involving OFA/iLRP through actions that may be independent of, or in conjunction with, the immune system. For example, the peptide G region of OFA/iLRP has been shown to play a role in metastasis through the stabilization of the laminin receptor [16, 24, 44]. The peptides listed in Table 1, including their mutated and/or modified forms may be used as a pharmacological agent by affecting OFA/iLRP activity.

In one embodiment, the present invention provides a test for determining the pharmacological effect of the growth rate on mammalian and non-mammalian cells. The test includes the steps of growing the cells with and without the peptides of the present invention at various concentrations, and measuring the effect on apoptosis, necrosis, and cell proliferation. OFA/iLRP-positive cancer cells can be grown in vitro on different basement membranes with the peptides of the present invention at various doses. The effect of the peptides can be measured by different methods including, but not limited to, DNA ladder, cell death detection ELISA, caspase measurement, TUNEL assay, Annexin-V membrane alterations, DNA stain, FAS, p53, cytotoxicity assay, cell proliferation, and cell viability.

The peptides may be used to increase or decrease the invasiveness of a OFA/iLRP-positive cancer cell. This can be measured by growing OFA/iLRP-positive cells at various concentrations, with and without peptides, using a modified Boyden-chamber similar to several studies involving other proteins [45-48]. The peptides may also be used to affect cell adhesion and can be measured using standard methods. For example, adherent cultured OFA/iLRP-positive cancer cells are cultured in the presence of different extra-cellular matrix proteins (ECM) and with the peptides. The cells are then assayed via standard methods to determine the relative attachment of the cell lines in the presences of the peptides [49-52]. Several other commonly used techniques may be applied to determine the affect of OFA/iLRP on cell viability, proliferation, cell death, and apoptosis [37-40].

### Use of Peptides for Monitoring of OFA/iLRP Associated Diseases

The peptides of the present invention can be used to monitor an ex-vivo or in vitro response to OFA/iLRP related disease. For example, the peptides listed in Table 1, individually or in combination, in a conjugated or unconjugated form, may be used to determine the extent of a body's response to disease treatment. The peptides listed in Table 1 can be coated onto a solid substrate (alone or in combination) and the cellular response, the presence of autoimmune antibodies, the presence of binding proteins, and/or other tests can be used to determine the response to dendritic cell therapy.

### Use of Peptides For Epitope Detection and Immunoglobulin Quantification

The peptides can be used as a substrate for in vitro epitope detection and quantification of immunoglobulin. The epitopes of OFA/iLRP listed in Table 1 can be coated on latex beads or particles and can be used to screen patients’ serum using agglutination. Briefly, the peptides listed in Table 1. can be attached to a particle such as latex or a colloid. The peptide/particle mixture can be incubated with patients' serum to measure the relative amounts of immunoglobulin present that react against OFA/iLRP peptide. If only one peptide reagent was used to sensitize dendritic cells, then that peptide is the only one required. If the full-length protein reagent is used to sensitize the dendritic cells, then a range of peptides can be predicted as the peptides listed in Table 1. To decrease the total number of peptide areas that cluster, MHC peptide prediction can be combined as described above. These peptides are conjugated individually to a latex bead, colloid, or particulate, and used for agglutination studies. To determine reactivity, the patients' serum, in a diluted or undiluted state, is transferred to a serological glass dish with a ˜25 mm diameter wax circle or on a platform specifically designed as a serology incubation template. The serum can be incubated with an appropriate amount of peptide/particulate mixture. The serum, peptide/particulate mixture should be constantly rotated on a horizontal platform to prevent non-specific agglutination. The test may include a positive serum and a negative control sample. After incubating from 15 min. to 2 hours and rotating at room temperature, the agglutination reaction is read and graded based upon the extent of agglutination. In certain cases, an additional anti-human antibody may be added to increase the sensitivity and detection rate of non-IgM molecules. This is an extension of the agglutination test described above and referred to as “indirect agglutination.” Additionally, the ratio of IgM (direct agglutination) to IgG (passive agglutination) may provide clinically relevant information on the patients' vaccination status. This technique may be applied to any dendritic cell therapy or cancer vaccine where a patient's immunity against specific epitopes needs to be rapidly determined and interpreted in reference to a grading scale. As an alternative to agglutination-scoring, the test may employ spectrophotometer, chemiluminescent, radioactive, electrical, or fluorescent quantification. Several other commonly used techniques may be applied to determine the immunological responses of OFA/iLRP and OFA/iLRP peptide therapy [37-40].

### Use of Peptides in Enzyme-Linked Immunosorbant Assays (ELISA)

The peptides of the present invention may be used in an ELISA assay. The ELISA assay can be used to quantify and/or detect soluble antigen or antibody. Additionally, different peptides are used to make specific individual responses to the different OFA/iLRP peptides. The first application is to use the peptide to act as an antigen against patient serum receiving OFA/iLRP therapy. This method follows standard protocols [40]. The second method would be another novel application of the peptides as a standard direct competitive assay to determine the circulating antigen. This method follows standard methods, but uses the peptides of the present invention [40].

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Example 1

### Peptide Predictions and Binding to OFA-Sensitized Dendritic Cells

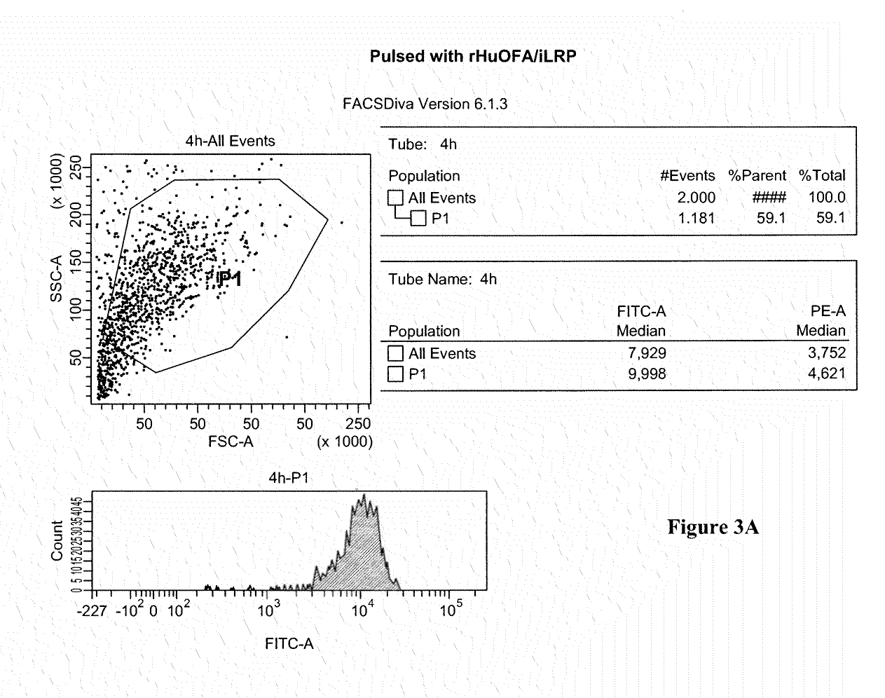

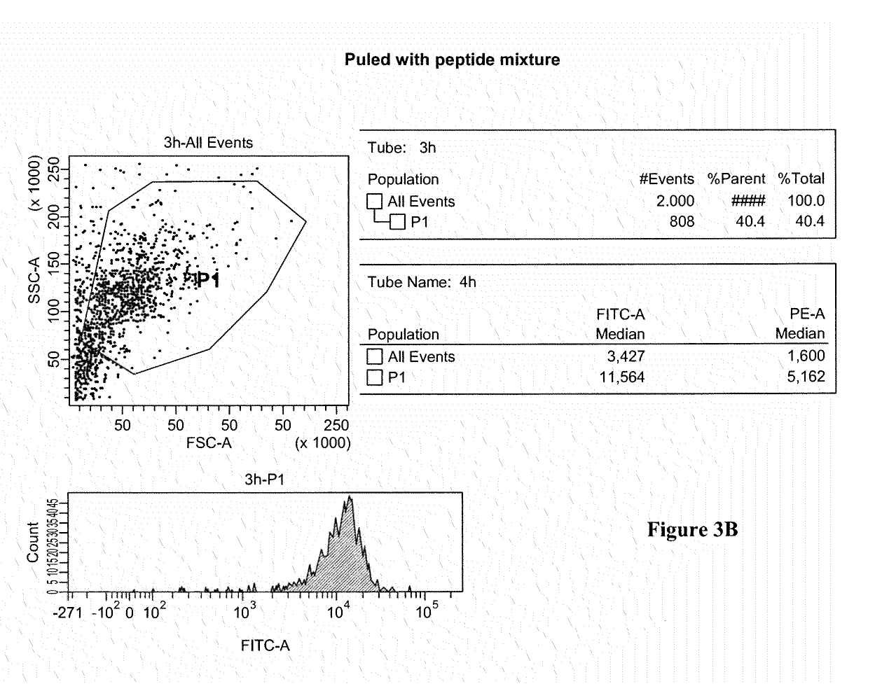

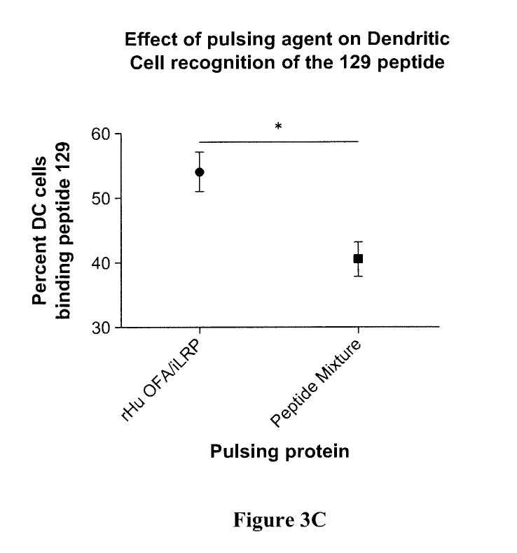

Several regions were predicted using the computer-based clustering methods (FIG. 2). The region starting at amino acid 129 (Ac-CADHQPLTEASYVNLPT-amide) was chosen because it can be used to further demonstrate the effect of peptide modifications. Additionally, 129 is in a region that was shown not to contain an epitope and lacks half of the YVNLPTIAL epitope shown to be required by previous studies {Rohrer, 2006 #1}. To determine if the 129 region is antigenic, dendritic cells were pulsed with full-length recombinant human OFA, matured and analyzed for 129 epitope using a fluorscein-labeled peptide (FIG. 3A & C).

CD 14+ monocytes were grown in serum-free dendritic cell medium containing: 1000 IU/ml GM-CSF and 1000 IU/ml of IL-4 (Cell Genix Antioch, IL) at 1×10⁶ cells per ml. The cells were pulsed with rHu OFA at 100 ng/ml or an equal mixture of the peptides (20 ng/ml) of:

| | |
|---|---|
| (129) | CPRADHQPLTEASYVNLPT —OH; |
| (117) | FREPRLLVVTDPRADHQPLTEAC-amide; |
| (101) | CGRFTPGTFTNQIQAAFREPT —OH; |
| (208) | Ac-EEIEKEEQAAAEKAVTKEEFQGC-amide |
| (54) | TWEKLLLAARAIVAIENPADVC-amide |

for 36 hours. The pulsed dendritic cells were matured using serum-free dendritic cell medium containing: 10 ng/ml IL-1beta, 1000 IU/ml IL-6, 5 ng/ml TNF-alpha with 1 μm prostaglandin E2 and matured for 2 days. At the end of two days, the cells were scraped from the plate and analyzed using flow cytometry analysis (FIGS. 3A-B).

Fluorescent labeling of the epitope starting at amino acid 129 was done following standard procedures, for example, the peptide that was first labeled on the cysteine was added to allow for conjugation using Fluorsceine-5-maledimide (Pierce/Thermo, Rockford, Ill.) following standard protocols. The unbound fluorsceine was removed using a standard dye-removal column (Pierce/Thermo, Rockford, Ill.).

To determine if the maturation was successful, the cells were analyzed with CD14, CD80/86, and Class II MHC (HLA-DR) following standard prototocols (R&D systems; Minneapolis, Minn.). Briefly, around 5.0×10⁵ cells per tube were re-suspended in phosphate-buffered saline with 2% fetal calf serum. The cells were placed on ice and 25 μl of the appropriate antibody, fluorsceine-labeled peptide or isotype-control antibodies was incubated with the cells on ice for 1 hour. After one hour, 4 ml of PBS with 2% FCS was added to each tube and the cells were pelleted by centrifuging tubes at 300×g for 10 min. The cells were resuspended in 400 μl of PBS with 2% FCS and analyzed (FIG. 3B). If the cells could not be analyzed immediately, they were fixed in 0.5% formaldehyde/PBS at 4° C. in the dark. Cells were considered to be dendritic cells if they were CD14 negative and CD83, CD80/86 and Class II MHC positive.

FIG. 3 shows representative FACS analysis of dendritic cells pulsed with full-length recombinant human OFA/iLRP (FIG. 3A) or an equal (by weight) mixture of the peptides (FIG. 3B) derived from the computational HLA analysis of OFA/iLRP. The data shows that both the rHu OFA/iLRP and the peptides can effectively pulse dendritic cells. The human rHu OFA/iLRP sensitized cells were recognized by 53.90±3.058 N=4 whereas the peptide mixture pulse recognized 40.43±2.618 N=4, for a difference of 13.48±4.025 (FIG. 3C). A two-tailed student t-test was used to determine if there were significant differences between the percentages of mature dendritic cells that recognized the 129 region of OFA/iLRP. The differences between the groups were found to be significant (p=0.0155); the mean+/− SEM and significance (*) is shown in FIG. 3C.

The data indicates that 53.9% of dendritic cells pulsed with rHu OFA/iLRP recognized the 129 region epitope (FIG. 3A) and that the computational analysis was accurate at predicting previously unidentified epitopes. As additional evidence to the strength of the region, the majority of the pulsed dendritic cells recognized this region. The ability of the peptides to stimulate dendritic cells is shown in FIG. 3C, as 40.43% of cells recognized the 129 regions. It would be expected that around 40% of the dendritic cells should recognize the 129 regions since the 117 peptide has overlapping sequences indicated by the underlined regions. This indicates that pulsing dendritic cells with OFA/iLRP peptides can be used to modulate immune responses to specific regions of the full-length protein. Additionally, these experiments indicate that fluorescent forms of the peptides can be used as both a sensitizing and a diagnostic reagent.

### Example 2

### Effect of Peptide Modifications on Dendritic Cell Binding of the 129 Regions

Several regions were predicted using the computer-based clustering methods (FIG. 2). The region starting at amino acid 129 (Ac-CADHQPLTEASYVNLPT-amide) was chosen because it can be used to further demonstrate the effect of peptide modifications. To determine if the 129 region can improve its relative binding to dendritic cells, the peptide sequence was modified on the carboxyl end to include 4 additional amino acids and the amino end included an 6-aminohexanoyl as a spacer to minimize binding hindrances. These modifications should increase the median fluorescent intensity when compared to the standard 129 peptide.

CD 14+ monocytes were grown in serum-free dendritic cell medium containing: 1000 IU/ml GM-CSF and 1000 IU/ml of IL-4 (Cell Genix Antioch, IL) at 1×10⁶ cells per ml. The cells were pulsed with rHu OFA at 100 ng/ml for 36 hours. The pulsed dendritic cells were matured using serum-free dendritic cell medium containing: 10 ng/ml IL-1beta, 1000 IU/ml IL-6, and 5 ng/ml TNF-alpha with 1 μm prostaglandin E2 and matured for 2 days. At the end of two days, the cells were scraped from the plate and analyzed using flow cytometry analysis.

Fluorescent labeling of peptides 129 and 129-a (Ac-TDPRADHQPLTEASYVNLPT-Ahx-C-amide) was done following standard procedures. Briefly, the peptide that was labeled on a cysteine included allowing for conjugation using Fluorsceine-5-maledimide (Pierce/Thermo, Rockford, Ill.) following standard protocols. The unbound fluorsceine was removed using a standard dye-removal column (Pierce/Thermo, Rockford, Ill.). This cysteine can also be used to conjugate the peptides to a range of different products.

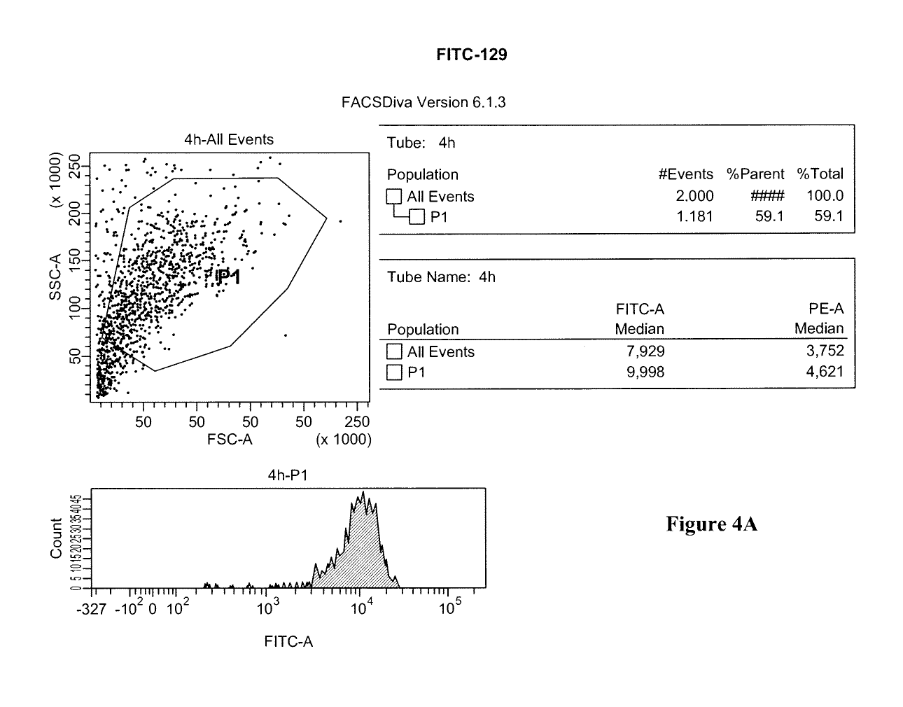

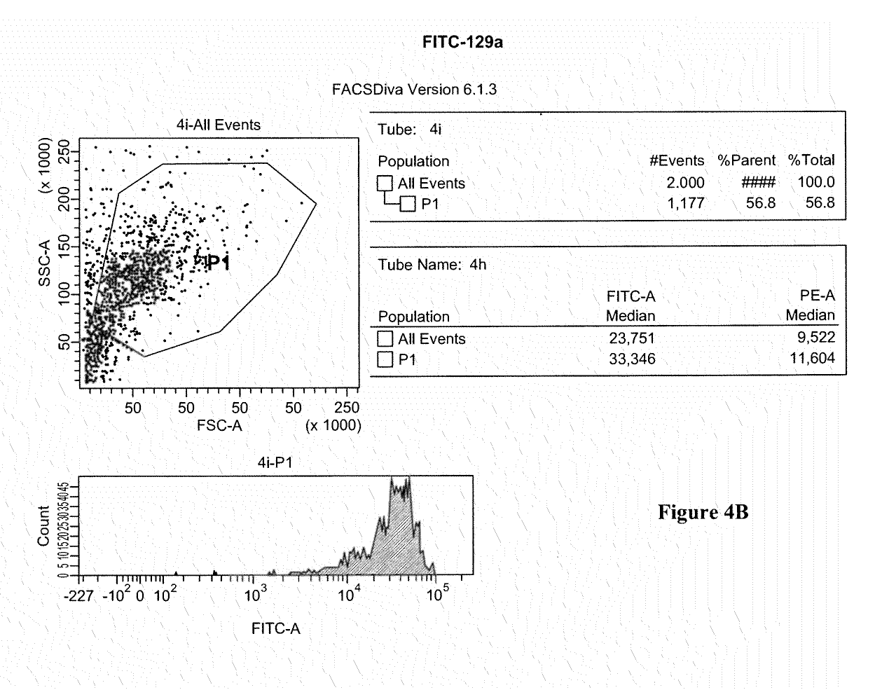

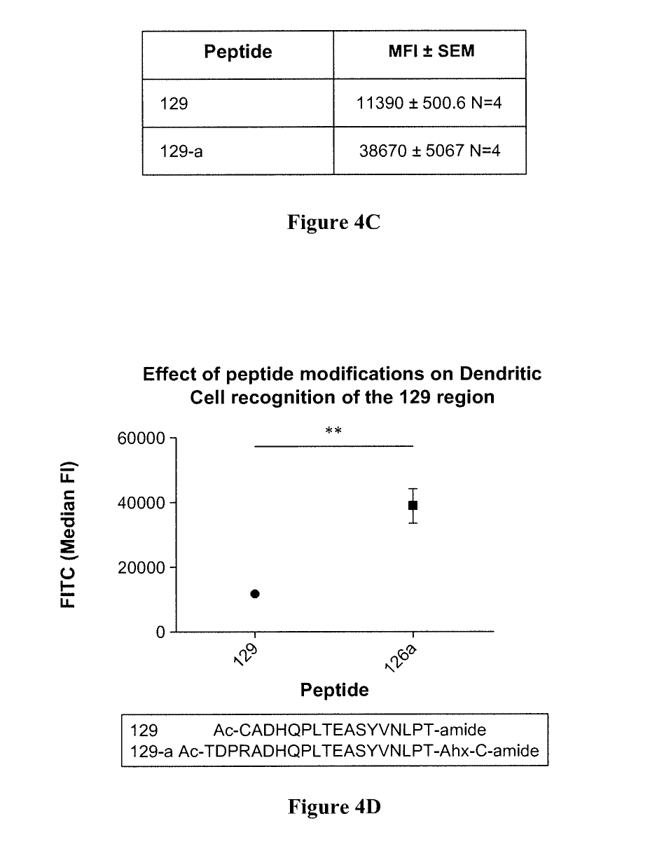

To determine if the maturation was successful, the cells were analyzed with CD14, CD83, CD80/86, and Class II MHC (HLA-DR) following standard protocols (R&D systems; Minneapolis, Minn.). Briefly, 0.5×10⁵ cells per tube were suspended in 25 μl or phosphate buffered saline with 2% fetal calf serum. The cells were placed on ice and 25 μl of the appropriate antibody, fluorsceine-labeled peptide or isotype control antibodies was incubated with the cells on ice for 1 hour. After one hour, 4 ml of PBS with 2% FCS was added to each tube and the cells were pelleted by centrifuging tubes at 300×g for 10 min. The cells were resuspended in 400 μl of PBS with 2% FCS and analyzed. If the cells could not be analyzed immediately, then they were fixed in 0.5% formaldehyde/PBS at 4° C. in the dark. At least 2,000 events were analyzed using a BD LSR II analyzer and displayed using FACSDiva software Version 6.1.3 (FIG. 4A-B). Cells were considered to be dendritic cells if they were CD14 negative and CD83, CD80/86, and HLA-DR positive.

FIG. 4D shows that dendritic cells pulsed with recombinant human OFA/iLRP are active against the 129 epitope and bind the modified 129-a with greater probability. The 129 peptide had a mean fluoresence intesity (MFI) of 11,390+/−500.6, whereas the 129-a peptide showed and MFI of 38,670+/−5067, or a 3.4-fold increase in fluorescent intensity. A representative of the 129 and 129-a FITC-labeled peptides are show in FIGS. 4A and B, respectively, determining if the 129-a has a different binding profile than 129 by t-test (p=0.0032), indicating that modifications to the peptide increased its fluorescent intensity through' either the use of the linker residue (Ahx) or the additional peptides (FIG. 4D). This increase MFI is indicative of increased binding capability.

This data demonstrates that slight changes can affect the binding and fluorescent intensity of the peptides. The peptides can be used to sensitize dendritic cells as well as todetermine the efficiency of dendritic cell pulsing. This data indicates that the use of the peptides designed against the OFA/iLRP protein can be slightly modified via a range of different methods to improve pulsing efficiency, binding activity, immune stimulation, and immune cell quantification. Peptides can be used alone or in a mixture to determine how reactive the immune cells/proteins are against specific OFA antigens.

### Example 3

### Innocyte 96-Well Cell Adhesion Assay

The goal of this experiment was to determine if the peptides designed against OFA/iLRP had any effect on cell adhesion of an adherent cell line to extracellular matrix components. The desired response is that there is no change in adhesion since decreased adhesion could increase metastatic potential.

All cells were grown in RPMI 1640 with L-glutamine, 100 I.U. Penicillin, 100 μg/ml Streptomycin, and 10% fetal calf serum at 37° C. in a humid chamber (Mediatech, Inc. Manassas, Va.). DU-145 AND SK-MEL-28 cells were obtained from American Type Culture Collection (ATCC, Manassas, Va.) and grown in media following standard protocols. Cells were grown to between 75 and 85% density and collected following standard protocols and counted using a modified Neubauer brightline hemacytometer and suspended at 400,000 cell/ml.

Peptides were dissolved in either phosphate buffered saline (PBS) or DMSO and then PBS to a maximum of 20% DMSO and a 2× solution was made and placed into complete medium. After the peptides were diluted to appropriate concentrations, 50 μl was dispensed into the 96-well assay plate provided in the kit supplied as 2×8-well strips coated with laminin I, fibronectin, vitronectin, collagen I, collagen III, and collagen IV (EMD, Gibbstown, N.J., along with 50 μl of cells (20,000 cells/well), and then incubated in a cell culture incubator for 2 hours at 37° C. The contents were shaken out into a biological waste container and gently washed by adding 200 μl of PBS to each well and shaking the contents out into the waste container with this step being repeated for a total of two washes. Next, 100 μl of Calcein-AM working solution was added to each well and incubated for 1 hour at 37° C. in an incubator. Fluorescence was measured in each well using a DTX 880 (Beckman Inc.) following standard fluorescent protocols with an excitation of 485 nm and an emission wavelength of 520 nm.

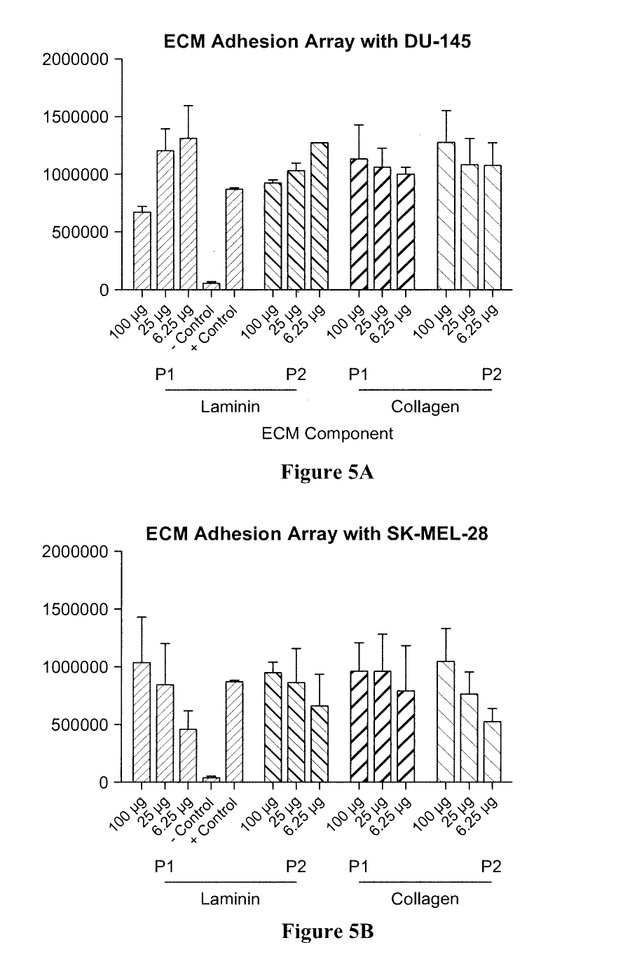

FIG. 5 shows data from the laminin I and collagen I wells. No significant differences are seen in adhesion of these cells in the presence of peptides in bioactive concentrations. Data is similar for all matrices (not shown).

This data indicates that designed OFA/iLRP peptides do not affect cell adhesion. Therefore, since adhesion is not affected, they should not increase metastatic load by decreasing the adhesion of cancer cells. This means that the peptides can have in vivo clinical/therapeutic uses that should not increase metastatic load of patients.

### Example 4

### The Effect of OFA/iLRP Peptides on Cell Viability

The goal of this experiment was to determine if the peptides designed against OFA/iLRP have any affect on cell viability. Due to the nature of OFA/iLRP, it was expected that peptides are designed to disrupt the OFA|OFA to LR conversion or inhibit other protein I protein interactions.

All cells were grown in RPMI 1640 with L-glutamine; 100 I.U. Penicillin, 100 μg/ml Streptomycin, and 10% fetal calf serum at 37° C. in a humid chamber (Mediatech, Inc. Manassas, Va.). DU 145 cells were obtained from American Type Culture Collection (ATCC, Manassas, Va.) and grown in media following standard protocols. DU145 cells were grown to between 75 and 85% density and collected following standard protocols and counted using a modified Neubauer brightline hemacytometer and suspended at 400,000 cell/ml.

Peptides were dissolved in either phosphate buffered saline (PBS) or DMSO and then PBS to a maximum of 20% DMSO and a 2× solution was made and placed into complete medium. After being diluted to appropriate concentration, 50 μl was dispensed into a 96-well assay plate, either coated with laminin/entactin complex (50 μg/ml) or untreated (Black with clear bottom) (Corning Life Sciences, Corning, N.Y.), 50 μl of cells (20,000 cells/well), and were grown overnight. Cells then had 20 μl of CellTiter-Blue added ((Promega, Madison, Wis.)) and incubated for an additional 2 hours at 37° C. Cells were then read on a DTX-880 (Beckman Inc.) following standard fluorescent protocols with an integration time of 0.001 sec. To determine if caspase activity was induced, Caspase 3/7 activity was determined using ApoOne assay (Promega, Madison, Wis.). The data was exported to Excel and then to Prism 5.0 (GraphPad Software, Inc) where it was plotted, and analyzed for statistical differences between the background control (diluents used for the peptide) using a one-way ANOVA.

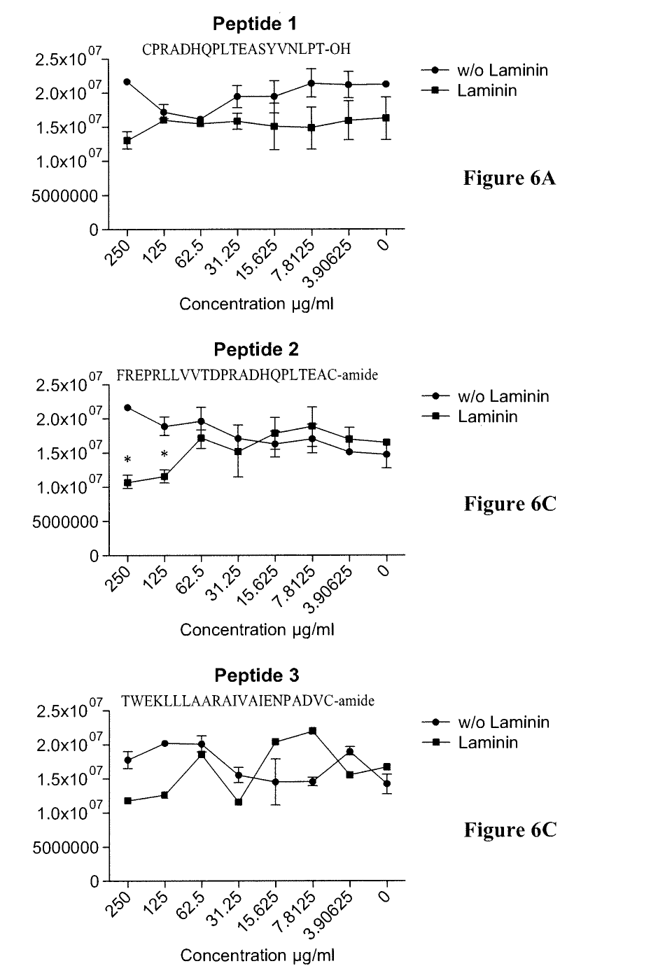

All of the DU145 cells grew on either the uncoated or laminin/entactin coated 96-well plates. In the presence of either peptide 1 or peptide 3 (Table 2), there was no statistical difference between the controls and the treatment group in either uncoated (w/o laminin) or coated (laminin) groups (FIGS. 6A & C). However, there was an interesting trend of any possible effect caused by the peptide requiring the presence of laminin. Peptide 2 (Table 2) showed the most significant biological effect with the ability to decrease cell viability by over 2-fold and was significantly different than the control (p<0.05) (FIG. 6B). No significant difference was seen in any group in the ApoOne Caspase 3/7 assay (data not shown). Additionally, a peptide made against the previously described OFA/iLRP epitope (amino acids 49-60 diluted in DMSO/PBS), while able to proliferate T-cells, was unable to affect cell viability or induce apoptosis (data not shown).

The goal of this experiment was to determine if any of the OFA/iLRP peptides predicted to have immunogenicity would have any effect on cell viability. When DU145 cells are grown in the presence of peptide 2, it appears to have a significant effect on cell viability when compared to peptide diluent control (20% DMSO/80% PBS). However, this was independent of Caspase 3/7 activation and no significant change in ApoOne assay was seen (data not shown). Additionally, peptides previously shown to be good epitopes (Rohrer, 2006 #1) did not affect cell viability (amino acids 49-60 data not shown). This indicates that the peptides may have therapeutic activity, which allows them to be used as either an immune or possible a cell viability target approach.

Many modifications and variations of the invention as hereinbefore set forth can be made without departing from the spirit and scope thereof, and therefore only such limitations should be imposed as are indicated by the appended claims.

All patent and literature references cited in the present specification are hereby incorporated by reference in their entirety.

TABLE 1
Initial screen of the putative OFA/iLRP
sequences for dendritic cell therapy.
The listed sequences may be used
alone or in conjunction for immune
stimulation for dendritic cell therapy
or other clinical applications.

| POS. | N | SEQUENCE | C | MW (Da) |
|---|---|---|---|---|
| 7 | ALD | VLQMKEEDV | LKF | 1072.24 |
| 9 | DVL | QMKEEDVLK | FLA | 1101.28 |
| 33 | LDF | QMEQYIYKR | KSD | 1240.45 |
| 45 | KSD | GIYIINLKR | TWE | 1071.33 |
| 57 | TWE | KLLLAARAI | VAI | 950.24 |
| 58 | WEK | LLLAARAIVA | IEN | 992.28 |
| 59 | EKL | LLAARAIVA | IEN | 879.12 |
| 60 | KLL | LAARAIVAI | ENP | 879.12 |
| 91 | LKF | AAATGATPI | AGR | 753.85 |
| 104 | GRF | TPGTFTNQI | QAA | 960.04 |
| 120 | REP | RLLVVTDPR | ADH | 1050.27 |
| 126 | VVT | DPRADHQPL | TEA | 1030.12 |
| 132 | ADH | QPLTEASYV | NLP | 989.1 |
| 133 | DHQ | PLTEASYVNL | PTI | 1088.23 |
| 177 | MWW | MLAREVLRM | RGT | 1100.41 |
| 183 | REV | LRMRGTISR | EHP | 1071.31 |
| 209 | DPE | EIEKEEQAA | AEK | 1028.1 |
| 211 | EEI | EKEEQAAAEK | AVT | 1114.19 |
| 212 | EIE | KEEQAAAEK | AVT | 985.07 |
| 213 | IEK | EEQAAAEKA | VTK | 927.98 |
| 215 | KEE | QAAAEKAVTK | EEF | 998.14 |
| 216 | EEQ | AAAEKAVTK | EEF | 870.01 |
| 254 | GVQ | VPSVPIQQF | PTE | 996.18 |

TABLE 2
Example of combining individual predicted
sites to generate one peptide. The above
five examples show how regions with in-
creased probability of immune and/or
cellular activity can be grouped together.
Slight modifications and the addition of
a cysteine at either end may be necessary
for conjugation.

| Number | Start site | Sequence |
|---|---|---|
| 1 | 54 | TWEKLLLAARAIVAIENPAD |
| 2 | 101 | GRFTPGTFTNQIQAAFREP |
| 3 | 117 | FREPRLLVVTDPRADHQPLTEA |
| 4 | 129 | ADHQPLTEASYVNLPTI |
| | 129-a | Ac-TDPRADHQPLTEASYVNLPT- |
| | | Ahx-C-amide |
| 5 | 208 | EEIEKEEQAAAEKAVTKEEFQ |

### REFERENCES

1. Lesot, H., U. Kuhl, and K. V. Mark, Isolation of a laminin-binding protein from muscle cell membranes. EMBO J, 1983. 2(6): p. 861-865.

2. Malinoff, H. L. and M. S. Wicha, Isolation of a cell surface receptor protein for laminin from murine fibrosarcoma cells. J Cell Biol, 1983. 96(5): p. 1475-9.

3. Rao, N. C., et al., Isolation of a tumor cell laminin receptor. Biochem Biophys Res Commun, 1983. 111(3): p. 804-8.

4. Biragyn, A., et al., Tumor-associated embryonic antigen-expressing vaccines that target CCR6 elicit potent CD8+ T cell-mediated protective and therapeutic antitumor immunity. J Immunol, 2007. 179(2): p. 1381-8.

5. Castronovo, V., et al., Immunodetection of the metastasis-associated laminin receptor in human breast cancer cells obtained by fine-needle aspiration biopsy. Am J Pathol, 1990. 137(6): p. 1373-81.

6. Coggin, J. H., Jr., A. L. Barsoum, and J. W. Rohrer, 37 kiloDalton oncofetal antigen protein and immature laminin receptor protein are identical, universal T-cell inducing immunogens on primary rodent and human cancers. Anticancer Res, 1999. 19(6C): p. 5535-42.

7. Coggin, J. H., Jr., J. W. Rohrer, and A. L. Barsoum, A new immunobiological view of radiation-promoted lymphomagenesis. Int J Radiat Biol, 1997. 71(1): p. 81-94.

8. Coggin, J. H., Jr., J. W. Rohrer, and A. L. Barsoum, True immunogenicity of oncofetal antigen/immature laminin receptor protein. Cancer Res, 2004. 64(13): p. 4685; author reply 4685.

9. Coggin, J. H., Jr., et al., 44-kd oncofetal transplantation antigen in rodent and human fetal cells. Implications of recrudescence in human and rodent cancers. Arch Otolaryngol Head Neck Surg, 1993. 119(11): p. 1257-66.

10. Friedrichs, B., et al., Humoral immune responses against the immature laminin receptor protein show prognostic significance in patients with chronic lymphocytic leukemia. J Immunol, 2008. 180(9): p. 6374-84.

11. Giannopoulos, K. and M. Schmitt, Targets and strategies for T-cell based vaccines in patients with B-cell chronic lymphocytic leukemia. Leuk Lymphoma, 2006. 47(10): p. 2028-36.

12. Gussack, G. S., et al., Human squamous cell carcinoma lines express oncofetal 44-kD polypeptide defined by monoclonal antibody to mouse fetus. Cancer, 1988. 62(2): p. 283-90. 13. Holtl, L., et al., Immunotherapy of metastatic renal cell carcinoma with tumor lysate-pulsed autologous dendritic cells. Clin Cancer Res, 2002. 8(11): p. 3369-76.

14. Jackers, P., et al., Isolation from a multigene family of the active human gene of the metastasis-associated multifunctional protein 37LRP/p40 at chromosome 3p21.3. Oncogene, 1996. 13(3): p. 495-503.

15. Karpatova, M., et al., Shedding of the 67-kD laminin receptor by human cancer cells. J Cell Biochem, 1996. 60(2): p. 226-34.

16. Magnifico, A., et al., Peptide G, containing the binding site of the 67-kDa laminin receptor, increases and stabilizes laminin binding to cancer cells. J Biol Chem, 1996. 271(49): p. 31179-84.

17. Menard, S., E. Tagliabue, and M. I. Colnaghi, The 67 kDa laminin receptor as a prognostic factor in human cancer. Breast Cancer Res Treat, 1998. 52(1-3): p. 137-45.

18. Rohrer, J. W., et al., Human breast carcinoma patients develop clonable oncofetal antigen-specific effector and regulatory T lymphocytes. J Immunol, 1999. 162(11): p. 6880-92.

19. Rohrer, S. D., et al., Expression of 44-kilodalton oncofetal antigen as a premalignancy marker in X irradiation-induced murine T-cell, lymphoma. J Natl Cancer Inst, 1992. 84(8): p. 602-9.

20. Sanjuan, X., et al., Overexpression of the 67-kD laminin receptor correlates with tumour progression in human colorectal carcinoma. J Pathol, 1996. 179(4): p. 376-80.

21. Siegel, S., et al., In-vivo detectable antibodies directed against the oncofetal antigen/immature laminin receptor can recognize and control myeloma cells-clinical implications. Leukemia, 2008. 22(11): p. 2115-8.

22. Siegel, S., et al., Identification of HLA-A*0201-presented T cell epitopes derived from the oncofetal antigen-immature laminin receptor protein in patients with hematological malignancies. J Immunol, 2006. 176(11): p. 6935-44.

23. Siegel, S., et al., Induction of cytotoxic T-cell responses against the oncofetal antigen-immature laminin receptor for the treatment of hematologic malignancies. Blood, 2003. 102(13): p. 4416-23.

24. Taraboletti, G., et al., Enhancement of metastatic potential of murine and human melanoma cells by laminin receptor peptide G: attachment of cancer cells to subendothelial matrix as a pathway for hematogenous metastasis. J Natl. Cancer Inst, 1993. 85(3): p. 235-40.

25. Viacava, P., et al., The spectrum of 67-kD laminin receptor expression in breast carcinoma progression. J Pathol, 1997. 182(1): p. 36-44.

26. Wewer, U. M., et al., Altered levels of laminin receptor mRNA in various human carcinoma cells that have different abilities to bind laminin. Proc Natl Acad Sci USA, 1986. 83(19): p. 7137-41.

27. Zelle-Rieser, C., et al., Expression and immunogenicity of oncofetal antigen-immature laminin receptor in human renal cell carcinoma. J Urol, 2001. 165(5): p. 1705-9.

28. Jamieson, K. V., et al., Crystal structure of the human laminin receptor precursor. J Biol Chem, 2008. 283(6): p. 3002-5.

29. Rohrer, J. W., A. L. Barsoum, and J. H. Coggin, Jr., Identification of oncofetal antigen/immature laminin receptor protein epitopes that activate BALB/c mouse OFA/iLRP-specific effector and regulatory T cell clones. J Immunol, 2006. 176(5): p. 2844-56.

30. Thomas, R. and P. E. Lipsky, Human peripheral blood dendritic cell subsets. Isolation and characterization of precursor and mature antigen-presenting cells. J Immunol, 1994. 153(9): p. 4016-28.

31. Zhou, L. J. and T. F. Tedder, CD14+ blood monocytes can differentiate into functionally mature CD83+ dendritic cells. Proc Natl Acad Sci USA, 1996. 93(6): p. 2588-92.

32. Weissman, D., et al., Three populations of cells with dendritic morphology exist in peripheral blood, only one of which is infectable with human immunodeficiency virus type 1. Proc Natl Acad Sci USA, 1995. 92(3): p. 826-30.

33. Zhou, L. J. and T. F. Tedder, A distinct pattern of cytokine gene expression by human CD83+ blood dendritic cells. Blood, 1995. 86(9): p. 3295-301.

34. Zhou, L. J. and T. F. Tedder, Human blood dendritic cells selectively express CD83, a member of the immunoglobulin superfamily. J Immunol, 1995. 154(8): p. 3821-35.

35. Freudenthal, P. S. and R. M. Steinman, The distinct surface of human blood dendritic cells, as observed after an improved isolation method. Proc Natl Acad Sci USA, 1990. 87(19): p. 7698-702.

36. Snider, D. E., Jr., The tuberculin skin test. Am Rev Respir Dis, 1982. 125(3 Pt 2): p. 108-18.

37. Ausubel, F. M.; Short protocols in molecular biology: compendium of methods from Current protocols in molecular biology. 5th ed. 2002, [Hoboken, N.J.]: J. Wiley.

38. Bonifacino, J. S., Short protocols in cell biology : a compendium of methods from Current protocols in cell biology. 2004, Hoboken, N.J.: John Wiley. 1 v. (various pagings).

39. Coligan, J. E., Short protocols in protein science : a compendium of methods from Current protocols in protein science. 2003, [Hoboken, N.J.]: Wiley. 1 v. (various pagings).

40. Coligan, J. E., Short protocols in immunology : a compendium of methods from current protocols in immunology. 2005, Hoboken, N.J.: John Wiley & Sons. 1 v. (various pagings).

41. Barsoum, A. L. and J. H. Coggin, Jr., Immunogenicity of a soluble partially purified oncofetal antigen from murine fibrosarcoma in syngeneic mice. J Biol Response Mod, 1989. 8(6): p. 579-92.

42. Rohrer, J. W. and J. H. Coggin, Jr., CD8 T cell clones inhibit antitumor T cell function by secreting IL-10. J Immunol, 1995. 155(12): p. 5719-27.

43. Rohrer, J. W., et al., Differential recognition of murine tumor-associated oncofetal transplantation antigen and individually specific tumor transplantation antigens by syngeneic cloned BALB/c and RFM mouse T cells. J Immunol, 1994. 152(2): p. 754-64.

44. Castronovo, V., G. Taraboletti, and M. E. Sobel, Functional domains of the 67-kDa laminin receptor precursor. J Biol Chem, 1991. 266(30): p. 20440-6.

45. Aznavoorian, S., et al., Molecular aspects of tumor cell invasion and metastasis. Cancer, 1993. 71(4): p. 1368-83.

46. Carroll, D. K., et al., p63 regulates an adhesion programme and cell survival in epithelial cells. Nat Cell Biol, 2006. 8(6): p. 551-61.

47. Ekblom, P., P. Lonai, and J. F. Talts, Expression and biological role of laminin-1. Matrix Biol, 2003. 22(1): p. 35-47.

48. Hood, J. D. and D. A. Cheresh, Role of integrins in cell invasion and migration. Nat Rev Cancer, 2002. 2(2): p. 91-100.

49. Horwitz, A. F. and T. Hunter, Cell adhesion: integrating circuitry. Trends Cell Biol, 1996. 6(12): p. 460-1.

50. Hynes, R. O., The dynamic dialogue between cells and matrices: implications of fibronectin's elasticity. Proc Natl Acad Sci USA, 1999. 96(6): p. 2588-90.

51. Li, S., et al., Matrix assembly, regulation, and survival functions of laminin and its receptors in embryonic stem cell differentiation. J Cell Biol, 2002. 157(7): p. 1279-90.

52. Stupack, D. G., Integrins as a distinct subtype of dependence receptors. Cell Death Differ, 2005. 12(8): p. 1021-30.

53. Donnes, P. and A. Elofsson, Prediction of. MHC class I binding peptides, using SVMHC. BMC Bioinformatics, 2002. 3(1): p. 25.

54. Reche, P. A., J. P. Glutting, and E. L. Reinherz, Prediction of MHC class I binding peptides using profile motifs. Hum Immunol, 2002. 63(9): p. 701-9.

55. Coligan, J. E., Short protocols in protein science : a compendium of methods from Current protocols in protein science. 2003, [Hoboken, N.J.]: Wiley. 1 v. (various pagings).

56. Coligan, J. E., Short protocols in immunology : a compendium of methods from current protocols in immunology. 2005, Hoboken, N.J.: John Wiley & Sons. 1 v. (various pagings).

57. Rammensee, H., et al., SYFPEITHI: database for MHC ligands and peptide motifs. Immunogenetics, 1999. 50(3-4): p. 213-9.

1. An isolated peptide selected from the group consisting of VLQMKEEDV (SEQ ID NO: 1), QMKEEDVLK (SEQ ID NO: 2), QMEQYIYKR (SEQ ID NO: 3), GIYIINLKR (SEQ ID NO: 4), KLLLAARAI (SEQ ID NO: 5), LLLAARAIVA (SEQ ID NO: 6), LLAARAIVA (SEQ ID NO: 7), LAARAIVAI (SEQ ID NO: 8), AAATGATPI (SEQ ID NO: 9), TPGTFTNQI (SEQ ID NO: 10), RLLVVTDPR (SEQ ID NO: 11), DPRADHQPL (SEQ ID NO: 12), QPLTEASYV (SEQ ID NO: 13), PLTEASYVNL (SEQ ID NO: 14), MLAREVLRM (SEQ ID NO: 15), LRMRGTISR (SEQ ID NO: 16), EIEKEEQAA (SEQ ID NO: 17), EKEEQAAAEK (SEQ ID NO: 18), KEEQAAAEK (SEQ ID NO: 19), EEQAAAEKA (SEQ ID NO: 20), QAAAEKAVTK (SEQ ID NO: 21), AAAEKAVTK (SEQ ID NO: 22), VPSVPIQQF (SEQ ID NO: 23), and a mixture thereof, wherein said peptide can sensitize dendritic cells.

2. The peptide of claim 1, wherein the peptide includes up to three additional amino acids at its n-terminal, c-terminal or both respectively.

3. The peptide of claim 2, wherein the peptide is

| PRADHQPLTEASYVNLPT | (SEQ ID NO: 24) |
|---|---|
| (129), | |
| FREPRLLVVTDPRADHQPLTEA | (SEQ ID NO: 25) |
| (117), | |
| GRFTPGTFTNQIQAAFREPT | (SEQ ID NO: 26) |
| (101), | |
| EEIEKEEQAAAEKAVTKEEFQG | (SEQ ID NO: 27) |
| (208), | |
| TDPRADHQPLTEASYVNLPT | (SEQ ID NO: 28) |
| (129-a) | |
| or | |
| TWEKLLLAARAIVAIENPADV | (SEQ ID NO: 29) |
| (54). | |

4. The peptide of claim 1, wherein the peptide contains a cysteine or other functional group that allows for conjugation to carriers.

5. The peptide of claim 3, wherein the peptide contains a cysteine or other functional group that allows for the conjugation to carriers.

6. The peptide of claim 1, wherein the peptide is conjugated to a carrier that increases the peptide's immune stimulation, stability, and/or solubility.

7. The peptide of claim 6, wherein the carrier is selected from a group consisting of keyhole limpet hemocyanin (KLH), serum albumin, biological polymers, antibody, chemotherapy, carbon nano-tubes, microelectro/electrofluidic device, molecular machine, amino acid MAP polymer, dendromer, biologically active lipids, biologically active sugar molecules/polymers, and colloidal particles.

8. The peptide of claim 1, wherein the peptide is modified to include acetylation, fatty acidification, myristic acidification, palmytolylilation, benzyloxycarbonylation, abidation, p-Nitroanilide, AMC, succinylation, NHS, CMK/FMK, D-amino acids, dinitrobenzoylation, methylation, phosphorylation, AHX, SO3H2, octanoic acid, biotin, FITC, GAM, Dansyl, MCA, HYNIC, DTPA, cyclic formations, or a multiple antigenic peptide system (MAP).

9. An isolated peptide derived from the dimerization region of OFA/iLRP, wherein the peptide can sensitize dendritic cells.

10. A pharmaceutical composition comprising the peptide of claim 1.

11. A pharmaceutical composition comprising the peptide of claim 3.

12. The composition of claim 11, further comprising a pharmaceutically acceptable carrier.

13. A dendritic cell that is sensitized by the peptide of claim 1, or a mixture thereof.

14. A dendritic cell that is sensitized by the peptide of claim 3, or a mixture thereof.

15. A vaccine comprising the isolated peptide of claim 1.

16. A vaccine comprising the isolated peptide of claim 3.

17. A method of treating a subject with OFA/iLRP related cancer comprising the steps of (a) sensitizing dendritic cells with the peptide of claim 1 or a mixture thereof, (b) administering the sensitized dendritic cells to the subject in an amount that is sufficient to induce an immune response that decreases the progression of the OFA/iLRP positive cancer.

18. A method of treating a subject with OFA/iLRP related cancer comprising the steps of (a) sensitizing dendritic cells with the peptide of claim 3 or a mixture thereof, (b) administering the sensitized dendritic cells to the subject in an amount that is sufficient to induce an immune response that decreases the progression of the OFA/iLRP positive cancer.

19. A method of treating a subject with OFA/iLRP related cancer comprising administering the peptide of claim 1, or a mixture thereof, to the subject in an amount that is sufficient to decrease the progression of the OFA/iLRP related cancer.

20. The method of claim 19, wherein the peptide induces an immune response in the subject that decreases the progression of the OFA/iLRP related cancer.

21. A method of treating a subject with OFA/iLRP positive cancer comprising administering the peptide of claim 3 or a mixture thereof to the subject in an amount that is sufficient to decrease the progression of the OFA/iLRP positive cancer.

22. A method of determining the amount of an antibody against OFA/iLRP in a sample comprising:
(a) contacting the peptide of claim 1, or a mixture thereof with the sample under a condition that allows the antibody to bind to the peptide to form a complex,
(b) determining the amount of complex formed in the sample.

23. A method of determining the amount of an antibody against OFA/iLRP in a sample comprising:
(a) contacting the peptide of claim 3 with the sample under a condition that allows the antibody to bind to the peptide to form a complex,
(b) determining the amount of complex formed in the sample.

24. A method of monitoring the progress of a OFA/iLRP related cancer vaccination therapy in a subject comprising the steps of (1) administering the peptide of claim 1 or a mixture thereof to a site of the subject subcutaneously or intradermally in an amount that is sufficient to detect the immune response of the subject to the therapy, (2) monitoring the diameter of the reaction at the site of administration.

25. A method of monitoring the progress of a OFA/iLRP related cancer vaccination therapy in a subject comprising the steps of (1) administering the peptide of claim 3 or a mixture thereof to a site of the subject subcutaneously or intradermally in an amount that is sufficient to detect the immune response of the subject to the therapy, (2) monitoring the diameter of the reaction at the site of administration.

26. A method of ex vivo monitoring the progress of a OFA/iLRP related treatment in a subject, wherein the treatment induces a T-cell related response, a B-cell related response, or both responses, comprising the steps of (1) providing a biofluid of the subject that receives the treatment, (2) contacting the peptides of the present invention with the biofluid under a condition that allows the interaction of the peptides with the T-cell, the B-cell or the products generated by the T-cell or B-cell, (3) determining the amount of interaction by ELISA, ELISpot, fluorescent polarization, resonance, or FACS method.
